(19) United States
(12) Patent Application Publication
(10) Pub. No.: US 2004/0014106 A1
(43) Pub. Date: Jan. 22, 2004

(54) Electrical detection of DNA hybridization and specific binding events

(75) Inventors: Timothy Patno, Evanston, IL (US); Christopher Khoury, Chicago, IL (US)

Correspondence Address: Edward K. Runyan, McDonnell Boehnen Hulbert & Berghoff, 32nd Floor, 300 S. Wacker Drive, Chicago, IL, 60606, US

(73) Assignee: Nanosphere, Inc., Northbrook, IL

(21) Appl. No.: 10/437,753

(22) Filed: May 14, 2003

Related U.S. Application Data
(60) Provisional application No. 60/380,441, filed on May 14, 2002.

(51) Int. Cl. C12Q 1/68; G01N 33/53; G01F 1/64

(52) U.S. Cl. 435/6; 435/7.1; 205/777.5

(57) ABSTRACT

A method for detecting a target analyte having a first binding site and a second binding site. A substrate is provided having at least a first and a second patterned conductor, the first conductor being separated from the second conductor. The arrangement of the patterned conductors forms at least two substantially non-conducting gaps. The method may also include contacting to the substrate capture probes that bind specifically to the first binding site of the target analyte and providing electrically conductive nanoparticles having bound thereto binding sites that bind specifically to the second binding site of the target analyte. Then, contacting the substrate and the electrically conductive nanoparticles with the target analyte under hybridizing conditions will bind the target analyte to the substrate and to the electrically conductive nanoparticles. The electrically conductive nanoparticles between the conductors can thus be electrically detected. Detection can be improved by silver deposition of the nanoparticles.

This application claims the benefit of U.S. Provisional Application No. 60/380,441, filed May 14, 2002, which is hereby incorporated by reference in its entirety.

### FIELD OF THE INVENTION

This invention relates to methods of detecting target analytes such as nucleic acids, whether natural or synthetic, and whether modified or unmodified, and, more particularly, to electrical detection of nucleic acids and other target analytes.

### BACKGROUND OF THE INVENTION

Sequence-selective DNA detection has become increasingly important as scientists unravel the genetic basis of disease and use this new information to improve medical diagnosis and treatment. DNA hybridization tests on oligonucleotide-modified substrates are commonly used to detect the presence of specific DNA sequences in solution. The developing promise of combinatorial DNA arrays for probing genetic information illustrates the importance of these heterogeneous sequence assays to future science.

Typically, the samples are placed on or in a substrate material that facilitates the hybridization test. These materials can be glass or polymer microscope slides or glass or polymer microtiter plates. In most assays, the hybridization of fluorophore-labeled targets to surface bound probes is monitored by fluorescence microscopy or densitometry. However, fluorescence detection is limited by the expense of the experimental equipment and by background emissions from most common substrates. In addition, the selectivity of labeled oligonucleotide targets for perfectly complementary probes over those with single-base mismatches can be poor, limiting the use of surface hybridization tests for detection of single nucleotide polymorphisms. A detection scheme which improves upon the simplicity, sensitivity and selectivity of fluorescent methods could allow the full potential of combinatorial sequence analysis to be realized.

### SUMMARY

The present system, in one aspect, allows for robust electrical detection of DNA hybridization events and other specific binding events using an array of microfabricated planar electrodes. In one embodiment of the invention, at least three electrodes are used to detect DNA hybridization events.

In another aspect of the invention, the electrodes are designed to maximize the surface area where hybridization can be detected. In one embodiment, the electrodes are designed such that at least one electrode has at least three sides, with at least a portion of two of the sides proximate to another electrode (or electrodes), with two of the sides and the other electrode (or electrodes) being separated by a gap.

### BRIEF DESCRIPTION OF THE DRAWINGS

FIG. 1a shows a schematic of a 3″ wafer mask comprising 4 chip patterns;

FIG. 1b shows a process of wafer fabrication that my be used to create patterned electrodes;

FIG. 1c shows a highlighted section from FIG. 1a of one electrode pair showing interdigitated patterned electrodes;

FIG. 2a shows, in greater detail, one chip of the wafer of FIG. 1a, with dots in the middle of each pattern of electrodes to symbolize where a robotic arrayer may spot a capture strand;

FIG. 2b shows one chip of an alternate, interdigitated electrode embodiment;

FIG. 2c shows, in greater detail, a patterned electrode pair of the embodiment of FIG. 2b;

FIG. 2d is an enlarged photograph showing the detection region formed by the patterned electrodes of FIG. 2c “spotted” with capture strands;

FIG. 3 illustrates an alternative design of patterned electrodes;

FIG. 4 illustrates another alternative design of pattern electrodes;

FIG. 5 is a cross-sectional view of a pair of patterned electrodes and capture probes on a substrate;

FIGS. 6a and 6b are schematic diagrams illustrating systems for detecting DNA using single nanoparticles (6a) and using nanoparticle trees (6b) to bind to targets.

### DETAILED DESCRIPTION

Definitions

“Analyte,” or “Target Analyte” as used herein, is the substance to be detected in the test sample using the present invention. The analyte can be any substance for which there exists a naturally occurring specific binding member (e.g., an antibody, polypeptide, DNA, RNA, cell, virus, etc.) or for which a specific binding member can be prepared, and the analyte can bind to one or more specific binding members in an assay. “Analyte” also includes any antigenic substances, haptens, antibodies, and combinations thereof. The analyte can include a protein, a peptide, an amino acid, a carbohydrate, a hormone, a steroid, a vitamin, a drug including those administered for therapeutic purposes as well as those administered for illicit purposes, a bacterium, a virus, and metabolites of or antibodies to any of the above substances.

“Capture probe” as used herein, is a specific binding member, capable of binding the analyte, which is directly or indirectly attached to a substrate. One example of a capture probe include oligonucleotides having a sequence that is complementary to at least a portion of a target nucleic acid and may include a spacer (e.g, a poly A tail) and a functional group to attach the oligonucleotide to the support. Other examples of capture probes include antibodies, proteins, peptides, amino acids, carbohydrates, hormones, steroids, vitamins, drugs, including those administered for therapeutic purposes as well as those administered for illicit purposes, bacteria, viruses, and metabolites of or antibodies to any of the above substances bound to the support either through covalent attachment or by adsorption onto the support surface. Examples of capture probes are described, for instance, in PCT/US01/10071 (Nanosphere, Inc.) which is incorporated by reference in its entirety.

“Specific binding member,” as used herein, is a member of a specific binding pair, i.e., two different molecules where one of the molecules, through chemical or physical means, specifically binds to the second molecule. In addition to antigen and antibody-specific binding pairs, other specific binding pairs include biotin and avidin, carbohydrates and lectins, complementary nucleotide sequences (including probe and captured nucleic acid sequences used in DNA hybridization assays to detect a target nucleic acid sequence), complementary peptide sequences, effector and receptor molecules, enzyme cofactors and enzymes, enzyme inhibitors and enzymes, cells, viruses and the like. Furthermore, specific binding pairs can include members that are analogs of the original specific binding member. For example a derivative or fragment of the analyte, i.e., an analyte-analog, can be used so long as it has at least one epitope in common with the analyte. Immunoreactive specific binding members include antigens, haptens, antibodies, and complexes thereof including those formed by recombinant DNA methods or peptide synthesis.

“Test sample,” as used herein, means the sample containing a target analyte to be detected and assayed using the present invention. The test sample can contain other components besides the analyte, can have the physical attributes of a liquid, or a solid, and can be of any size or volume, including for example, a moving stream of liquid. The test sample can contain any substances other than the analyte as long as the other substances do not interfere with the specific binding of the specific binding member or with the analyte. Examples of test samples include, but are not limited to: Serum, plasma, sputum, seminal fluid, urine, other body fluids, and environmental samples such as ground water or waste water, soil extracts, air and pesticide residues.

“Type of oligonucleotides” refers to a plurality of oligonucleotide molecules having the same sequence. A “type of” nanoparticles, conjugates, etc. having oligonucleotides attached thereto refers to a plurality of that item having the same type(s) of oligonucleotides attached to them.

“Nanoparticles having oligonucleotides attached thereto” are also sometimes referred to as “nanoparticle-oligonucleotide conjugates” “nanoparticle conjugates”, or, in the case of the detection methods of the invention, “nanoparticle-oligonucleotide probes,” “nanoparticle probes,” “detection probes” or just “probes.” The oligonucleotides bound to the nanoparticles may have recognition properties, e.g., may be complementary to a target nucleic acid, or may be used as a tether or spacer and may be further bound to a specific binding pair member, e.g., receptor, against a particular target analyte, e.g, ligand. For examples of nanoparticle-based detection probes having a broad range of specific binding pair members to a target analyte is described in PCT/US01/10071 (Nanosphere, Inc.) which is hereby incorporated by reference in its entirety.

One detection technique that improves upon fluorescent methods is an electrical chip-based DNA detection method that employs detection probes. A probe may use synthetic strands of DNA or RNA that are complementary to specific target analytes. Attached to the synthetic strands of nucleic acid is a signal mechanism. If the signal is present (i.e., there is a presence of the signal mechanism), then the synthetic strand has bound to nucleic acid in the sample so that one may conclude that the target nucleic acid is in the sample. Conversely, the absence of a signal indicates that no target nucleic acid is present in the sample.

An example of a signal mechanism is a gold nanoparticle probe with a relatively small diameter (10 to 40 nm), modified with oligonucleotides, to indicate the presence of a particular DNA sequence hybridized on a substrate in a three-component sandwich assay format. See U.S. Pat. No. 6,361,944 entitled “Nanoparticles having oligonucleotides attached thereto and uses therefore,” herein incorporated by reference in its entirety; see also T. A. Taton, C. A. Mirkin, R. L. Letsinger, Science, 289, 1757 (2000). The selectivity of these hybridized nanoparticle probes for complementary over mismatched DNA sequences was intrinsically higher than that of fluorophore-labeled probes due to the uniquely sharp dissociation (or “melting”) of the nanoparticles from the surface of the array. In addition, enlarging the array-bound nanoparticles by gold-promoted reduction of silver permitted the arrays to be imaged in black-and-white by a flatbed scanner with greater sensitivity than typically observed by confocal fluorescent imaging of fluorescently labeled gene chips. The scanometric method was successfully applied to DNA mismatch identification.

It is a challenge to detect a binding event between complementary single-strands of DNA using an immobilized capture probe (such as, for example, an oligonucleotide) and a target analyte in combination with a conductive particle, such as a gold nanoparticle. Conductive particles, such as gold or other conductive or semiconducting nanoparticles, can create an electrically detectable bridge between two electrodes (or contacts) when the binding event occurs. Such a bridge changes the electrical characteristics between the two electrodes. For example, the bridge may change the electrical impedance characteristics (e.g., from high to low impedance), thus allowing for reliable measurement of changes in resistance or some other variable (such as capacitance, inductance, AC signals) using a readily available instrument such as a multimeter or an LCR meter.

Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂, S₃, In₂, Se₃, Cd₃P₂, Cd₃, As₂, InAs, and GaAs. The size of the nanoparticles is preferably from about 5 nm to about 150 nm (mean diameter), more preferably from about 5 to about 50 nm, most preferably from about 10 to about 30 nm.

Methods of making metal, semiconductor and magnetic nanoparticles are well-known in the art. See, e.g., Schmid, G. (ed.) Clusters and Colloids (V C H, Weinheim, 1994); Hayat, M. A. (ed.) Colloidal Gold: Principles, Methods, and Applications (Academic Press, San Diego, 1991); Massart, R., IEEE Taransactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988).

Methods of making ZnS, ZnO, TiO₂, AgI, AgBr, HgI₂, PbS, PbSe, ZnTe, CdTe, In₂ S₃, In₂, Se₃, Cd₃, P₂, Cd₃, As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky et al., J. Am. Chem. Soc., 112, 9438 (1990); Ushida et al., J. Phys. Chem., 95, 5382 (1992). Suitable nanoparticles are also commercially available from, e.g., Ted Pella, Inc. (gold), Amersham Corporation (gold) and Nanoprobes, Inc. (gold).

Gold colloidal particles have high extinction coefficients for the bands that give rise to their distinctive colors. These intense colors change with particle size, concentration, interparticle distance, and extent of aggregation and shape (geometry) of the aggregates, making these materials particularly attractive for colorimetric assays. For instance, hybridization of oligonucleotides attached to gold nanoparticles with oligonucleotides and nucleic acids results in an immediate color change visible to the naked eye. In addition, gold nanoparticles have excellent electrical conduction properties that make them particularly suitable for use with the present system. Semiconductor nanoparticles are also suitable for use in nanofabrication because of their unique electrical and luminescent properties.

The nanoparticles, the oligonucleotides, or both, are functionalized in order to attach the oligonucleotides to the nanoparticles. Such methods are known in the art. For instance, oligonucleotides functionalized with alkanethiols at their 3′-termini or 5′-termini readily attach to gold nanoparticles. See, for example, Whitesides, Proceedings of the Robert A. Welch Foundation 39th Conference On Chemical Research Nanophase Chemistry, Houston, Tex., pages 109-121 (1995). See also Mucic et al., Chem. Commun. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes).

Oligonucleotides terminated with a 5′ thionucleoside or a 3′ thionucleoside may also be used for attaching oligonucleotides to solid surfaces. Gold nanoparticles may be attached to oligonucleotides using biotin-labeled oligonucleotides and streptavidin-gold conjugate colloids; the biotin-streptavidin interaction attaches the colloids to the oligonucleotide. Shaiu et al., Nucleic Acids Research, 21, 99 (1993). The following references describe other methods that may be employed to attach oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langrnuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

Each nanoparticle may have a plurality of oligonucleotides attached to it, and as a result, each nanoparticle-oligonucleotide conjugate can bind to a plurality of target analytes having the complementary sequence. The present invention relates to the detection of metallic or conductive nanoparticles on the surface of a substrate. The substrate's surface may have a plurality of spots containing specific binding complements (i.e., capture probes) to one or more target analytes. One of the spots on the substrate may be a test spot (containing a test sample) for nanoparticles complexed thereto in the presence of one or more target analytes. Another one of the spots may be a control spot or second test spot. When testing for infectious diseases, for example, a control spot may be used (or control-positive and control-negative spots) to compare with the test spot in order to detect the presence or absence of a target analyte in the test sample. The target analyte could be representative of a specific bacteria or virus, for example. The control-positive spot may be a metallic nanoparticle conjugated directly to the substrate via a nucleic capture strand, metallic nanoparticles printed directly on the substrate, or a positive result of metallic nanoparticles complexed to a known analyte. A second test spot may be used when testing for genetic disposition (e.g., which gene sequence is present). For example, two test spots are used for comparison of gene sequences, such as single nucleotide polymorphisms.

Oligonucleotides of defined sequences are used for a variety of purposes in the practice of the invention. Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

The present system allows for electrically detecting target analytes. Any type of target analyte, such as nucleic acid or protein, may be detected, and the methods may be used for the diagnosis of disease or infection, identification of drugs or pollutants, or for sequencing of nucleic acids. Examples of nucleic acids that can be detected by the methods of the invention include genes (e.g., a gene associated with a particular disease), viral RNA and DNA, bacterial DNA, fungal DNA, CDNA, mRNA, RNA and DNA fragments, oligonucleotides, synthetic oligonucleotides, modified oligonucleotides, single-stranded and double-stranded nucleic acids, natural and synthetic nucleic acids, etc.

Thus, examples of the uses of the methods of detecting nucleic acids include: the diagnosis and/or monitoring of viral diseases (e.g., human immunodeficiency virus, hepatitis viruses, herpes viruses, cytomegalovirus, and Epstein-Barr virus), bacterial diseases (e.g., tuberculosis, Lyme disease, H. pylori, Escherichia coli infections, Legionella infections, Mycoplasma infections, Salmonella infections), sexually transmitted diseases (e.g., gonorrhea), inherited disorders (e.g., cystic fibrosis, Duchene muscular dystrophy, phenylketonuria, sickle cell anemia), and cancers (e.g., genes associated with the development of cancer); in forensics; in DNA sequencing; for paternity testing; for cell line authentication; for monitoring gene therapy; and for many other purposes.

The nucleic acid to be detected may be isolated by known methods, or may be detected directly in cells, tissue samples, biological fluids (e.g., saliva, urine, blood, serum), solutions containing PCR components, solutions containing large excesses of oligonucleotides or high molecular weight DNA, and other samples, as also known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). Methods of preparing nucleic acids for detection with hybridizing probes are well known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). If a nucleic acid is present in small amounts, it may be amplified by methods known in the art. See, e.g., Sambrook et al., Molecular Cloning: A Laboratory Manual (2nd ed. 1989) and B. D. Hames and S. J. Higgins, Eds., Gene Probes 1 (IRL Press, New York, 1995). One method of amplification is polymerase chain reaction (PCR) amplification.

Electrically detecting nucleic acids allows robust, high throughput detection which makes it particularly suitable for use in, e.g., research and analytical laboratories in DNA sequencing, in the field to detect the presence of specific pathogens, in the doctor's office for quick identification of an infection to assist in prescribing a drug for treatment, and in homes and health care centers for inexpensive first-line screening.

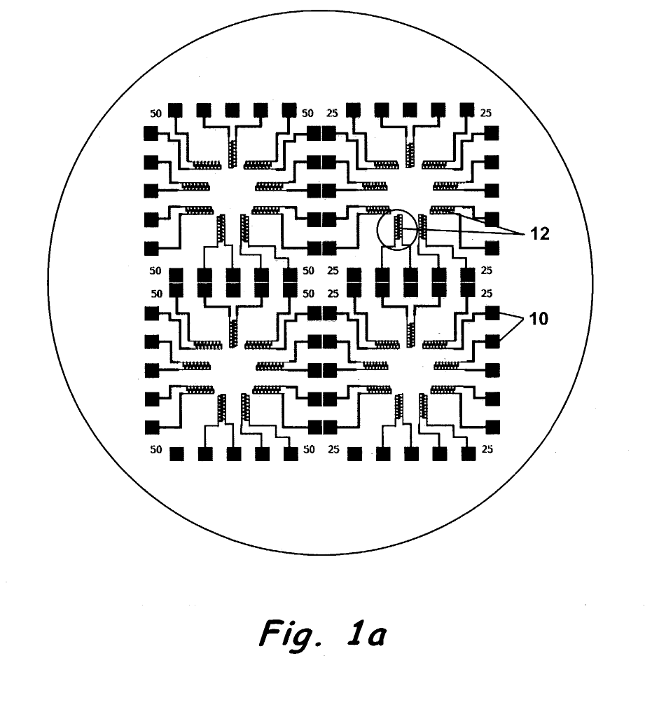

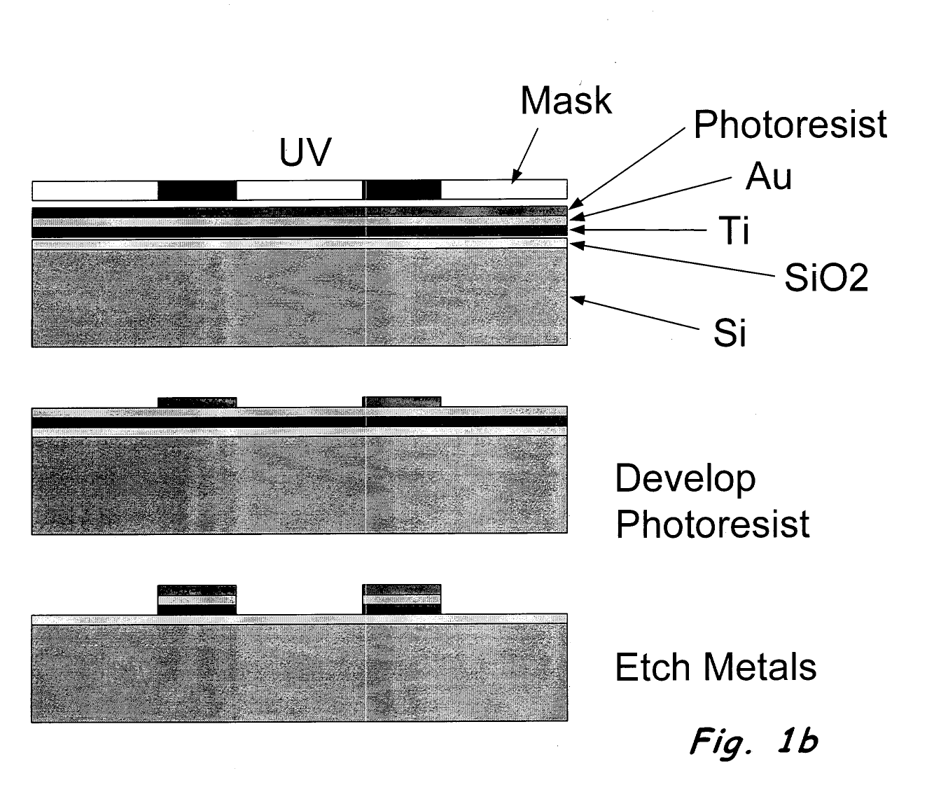

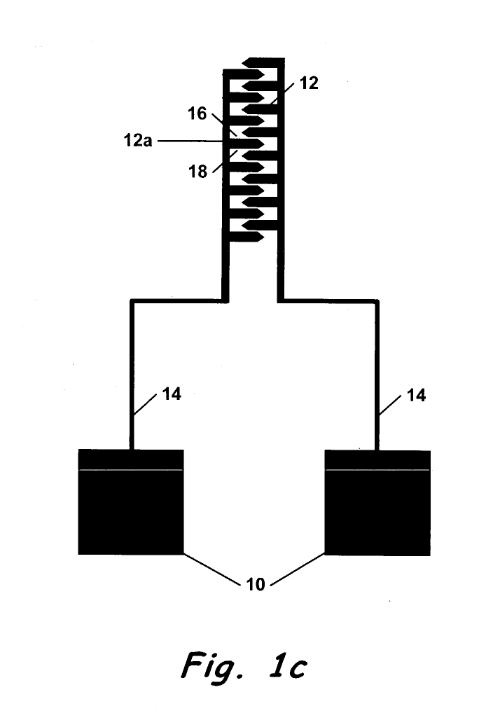

Referring now to the drawings, FIG. 1a is a layout of a 3″ wafer mask with 4 chip patterns on it, with each chip pattern having 10 electrical detection regions formed by complementary patterned conductors or electrodes, 12 and 12a. Each electrical detection region is suitable for detecting the presence of a nucleic acid. The size of the wafer mask and the number of chip patterns may depend on the criteria of the system. As shown in FIG. 1a, at least two contact pads 10 are provided for each detection region. The contact pads 10 are electrically connected to the electrodes 12 as shown. One example of such a pair of contact pads 10 and plurality of electrodes 12 are shown in FIG. 1b (which is the circled section in FIG. 1a).

An example of the process for making a 4-chip wafer on a glass substrate follows. First, the wafer and tools are cleaned with Acetone/IPA/Water/IPA/Nitrogen. Then, the wafer is Piranha cleaned (H₂SO₄:H₂O₂ 1:4) for 10 minutes and a layer of silicon dioxide is grown on the wafer's surface. Next, a 50 Å layer of Titanium and a 900 Å layer of Gold are deposited on the wafer using e-beam evaporation. Next, the wafer is hotplate baked for 5 minutes at 115 degrees C. to thoroughly dry it before spin-depositing 1.5 mL of photoresist (such as Shipley 1818) on the wafer at 5,000 rpm. The wafer is then hotplate baked again for 1 minute at 115 degrees C. to drive out any remaining resist solvent. Next, the wafer is aligned and exposed for 11 seconds, then developed for 1 minute. The wafer is then hotplate baked at 115 degrees C. for 2 minutes to harden the photoresist. Next the wafer is etched for 30 seconds (gold layer) and then for another 24 seconds (chromium layer) and rinsed and dried. Next, the photoresist is removed with a remover such as Shipley 1165, and the photoresist is further plasma stripped. The wafer is inspected for any residual photoresist, and is then diced between contact pads to create four chips. A very similar procedure is used for processing glass wafers. A cross-sectional outline of this process is shown in FIG. 1b.

As shown in FIG. 1c, there are a plurality of electrodes (with 16 electrodes in all). More or fewer electrodes may be used depending on the needs of the system. The electrodes may be arranged in an “interdigitated” pattern. Thus, the electrodes are meshed together, separated by a non-conductive gap. In some embodiments, it may be useful to pattern an insulator such as a nitride or oxide in the gap between electrodes. In one aspect, at least three electrodes are used. Two electrodes may be disposed in one direction and the third electrode may be disposed in the opposite direction.

As shown in FIG. 1c, the exemplary electrode has a plurality of sides (such as the 5 sided electrode in FIG. 1c), with at least one of the sides connected to the conductive trace 14. Moreover, the electrodes are placed such that at least one of the electrodes, such as the electrode designated as 12a, has at least two sides proximate to other electrodes, with two of the sides and the other electrode (or electrodes) being separated by a non-conductive gap. For example, sides 16 and 18 are proximate to other electrodes, separated by a non-conductive gap.

As discussed above, FIG. 1a shows a wafer mask having four chip patterns. Each chip may be designed to be geometrically compatible with an arrayer and microscope slide format. Three chips will fit on, or can comprise, one standard arrayer microscope slide. Because each chip includes a series of interdigitated electrodes that allow detection at any point within the detection region, there is a large amount of tolerance for the arrayer to place or “spot” capture probes on the region. Microfabrication allows for a denser array of electrodes and more consistent measurements.

The device may be fabricated in a clean room environment. The substrate may, for example, be a double-sided polished Silicon 3″ wafer, although any suitable substrate may be used. For example, the substrate may be composed of glass (e.g., a standard arrayer microscope slide) instead of silicon. An insulating layer, such as an oxide layer (SiO₂), may be grown on the wafer in a wet thermal environment, although an insulating layer is not necessarily critical to all embodiments of the apparatus. Other insulating materials include, but are not limited to silicon nitride and polyamide. Conductive layers, such as metal layers (e.g., gold, platinum, aluminum, chromium or copper), may be deposited on the wafer and patterned using a photolithography process. In an alternate embodiment, the conductive layer may include a semiconducting material.

Photolithography, chemical development and etching of the wafer results in the microfabricated electrodes. A high impedance exists between each electrode pair unless a conductive bridge is formed. Dicing of the wafers into individual 25 mm×25 mm squares results in a “chip” that may comprise multiple complementary sets of patterned electrodes capable of electrically detecting nanoparticles. For example, the wafer of FIG. 1a has four chip patterns, and each chip has 9 sets of patterned electrodes for sensing nanoparticles. Each chip is thoroughly cleaned of all organic materials in an oxygen plasma environment and is then passivated. Afterwards, the chip is spotted in an arrayer with capture probes, such as oligonucleotide capture strands.

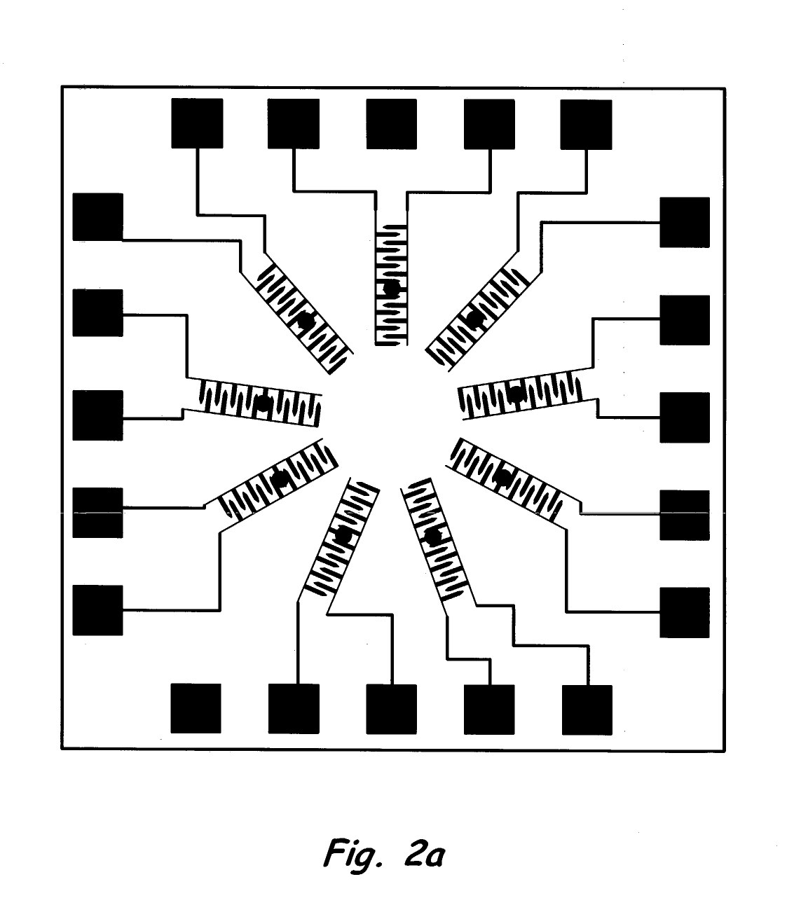

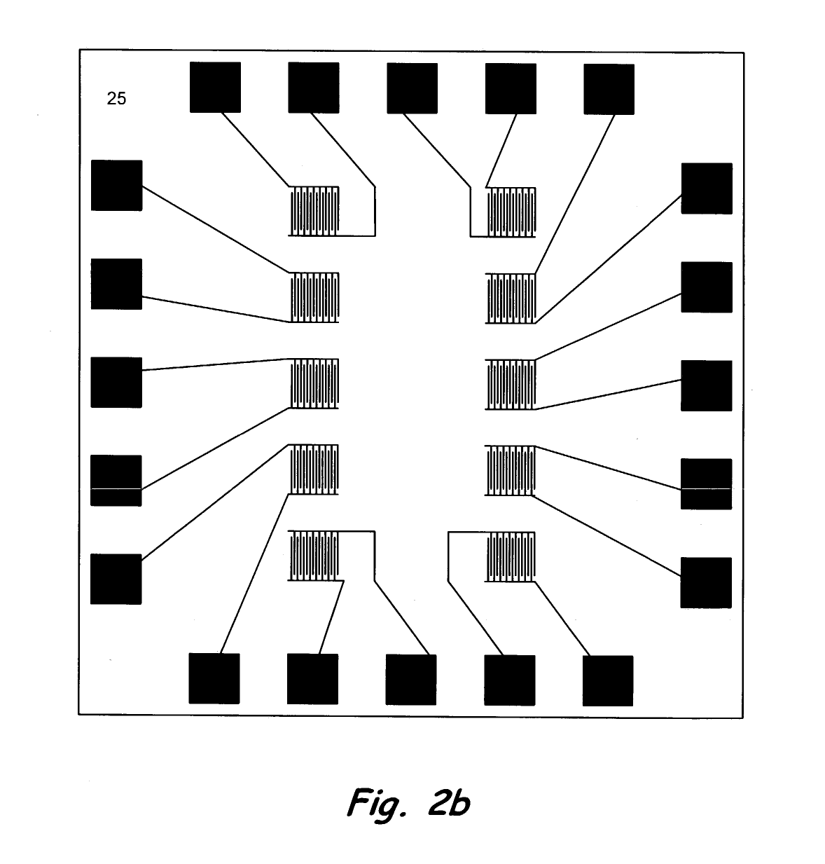

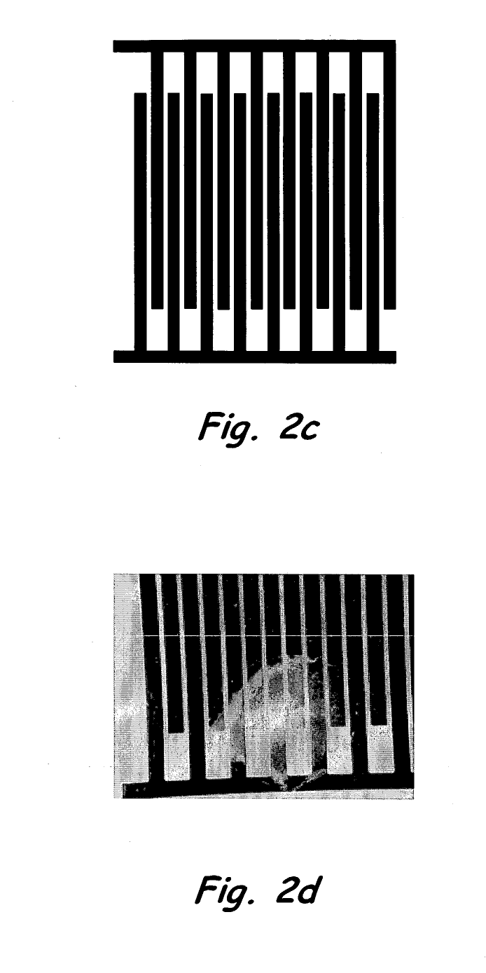

FIG. 2a illustrates an alternate embodiment of an evenly spaced electrode design. A robotic arrayer may dispense spots comprising one or more capture strands. FIG. 2 shows the dots in the middle of the figure as symbolizing where a robotic arrayer may “spot”, or place, a capture strand. Robotic arrayers, while automated, vary in the placement accuracy of dispensing capture strands. The spots have, for example, a typical location tolerance of ±1 mm. In the Figure, as long as an arrayer spots capture strands so that some of them are within the gaps between electrodes, electrical detection of nanoparticles bound (directly or indirectly) to the capture strands will be possible.

FIG. 2b shows an alternate embodiment of a chip with 10 sets of complementary, interdigitated electrodes. This embodiment results in a larger, square sensing region formed by the gaps between electrodes. A useful size of sensitive regions could be between 500 μm² and 2 mm², for example.

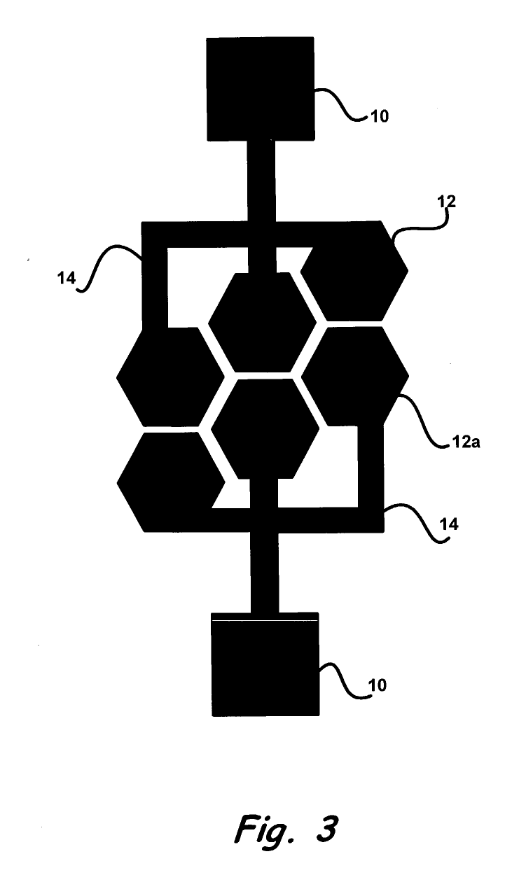

Because the patterned electrodes cover a much larger portion of the substrate than a single end-to-end gap formed by two electrodes, spotting with a robotic arrayer is possible despite placement errors inherent in robotic arrayers. Moreover, the geometry allows for multiple spots to be placed on a single chip, which can enhance detection reliability. Finally, concentration variations of capture strands within spots are possible. The electrode design accounts for any potential variations, since an entire spot, rather than just a portion of it, can be positioned within a detection region formed by the patterned electrodes. FIG. 3 shows alternate, hexagonally shaped electrodes 12 and 12a connected via conductive traces 14 to contact pads 10.

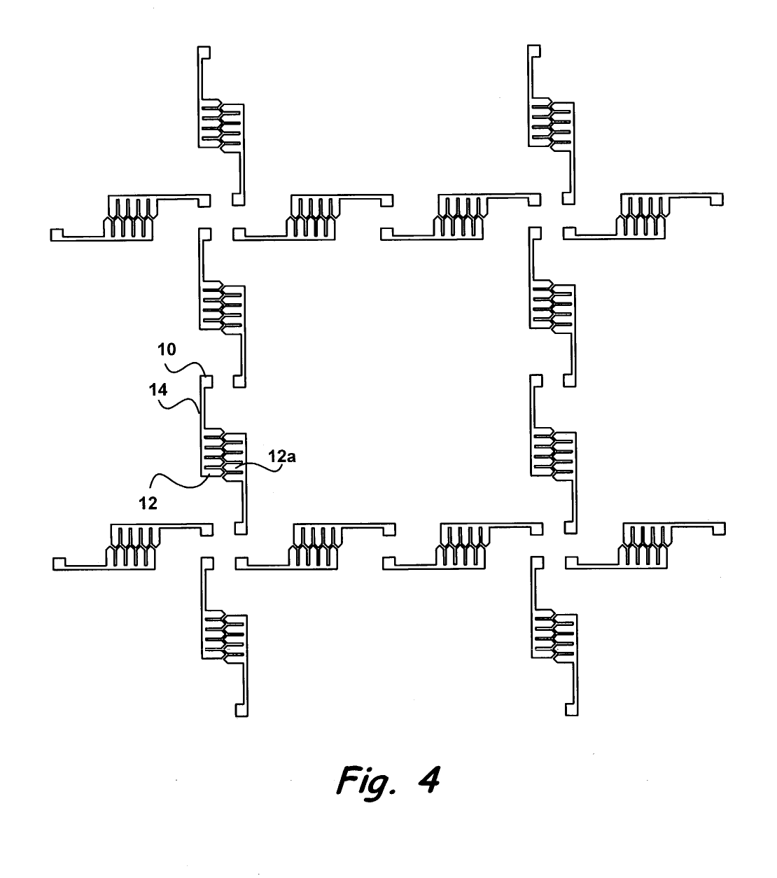

FIG. 4 illustrates another embodiment of the invention. Similar to the previous figures, electrodes 12 and 12a are connected to a contact pads 10 via conductive traces 14. The electrodes 12 and 12a, rather than being sandwiched in between one another, as shown in FIG. 1b, abut one another with a gap or an oxide layer between them. The particular configuration for the electrodes and contact pads allows for compact and high density geometries.

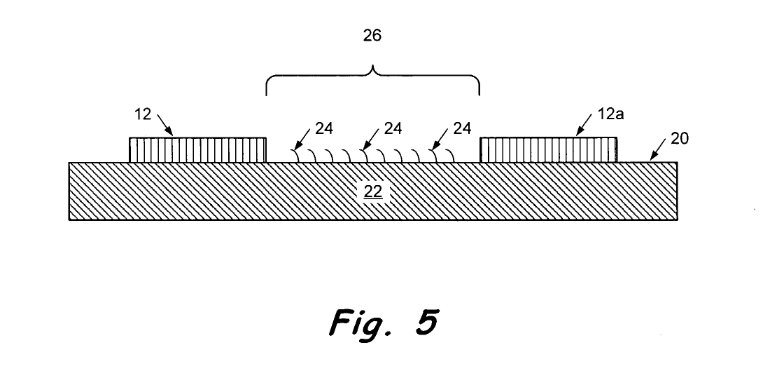

FIG. 5 illustrates a cross-section of electrodes 12 and 12a patterned on the surface 20 of a substrate 22. Capture probes 24 are immobilized within the substantially non-conducting gap 26 between electrodes 12 and 12a. When a binding event between matching single-strands of DNA using an immobilized capture probe 24, a target analyte in combination with a conductive particle occurs, the electrical characteristics between electrodes 12 and 12a measurably changes. For example, the gold nanoparticles of the detection probes can bridge the substantially non-conducting gap between the electrodes, increasing the conductance between the electrodes.

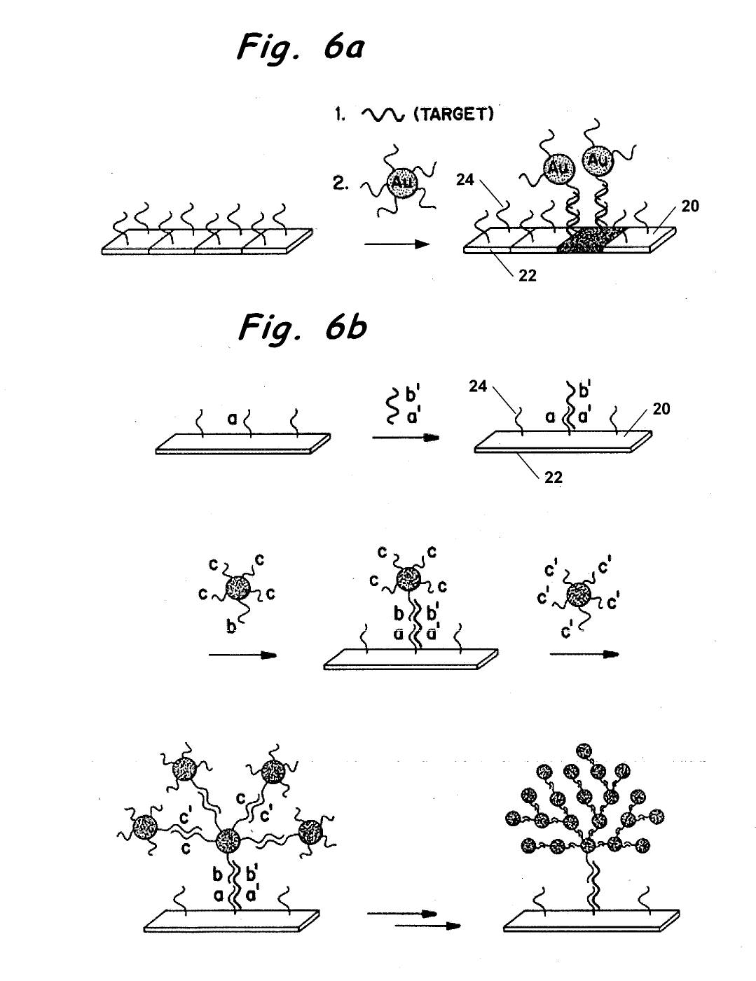

Note that the nanoparticles can either be individual ones or “trees” of nanoparticles bound to each other. Schematics illustrating detection of target analytes on a substrate are shown in FIGS. 6a and 6b. FIG. 6a shows target analytes binding individual gold nanoparticles to capture probes 24 that are immobilized on the surface 20 of substrate 22. FIG. 6b shows target analytes binding trees of nanoparticles to capture probes 24 that are immobilized on the surface 20 of substrate 22. In FIGS. 6a and 6b, a, b, and c refer to different binding sites (e.g., oligonucleotide sequences), whereas a′, b′, and c′ refer to binding sites, such as oligonucleotide sequences, that are complementary to a, b, and c, respectively.

The trees increase signal sensitivity as compared to individual nanoparticles, and the hybridized gold nanoparticle trees often can be observed with the naked eye as dark areas on a substrate. When nanoparticle trees are not used, or to further amplify the signal produced by the trees, the hybridized gold nanoparticles can be treated with a silver staining solution. The trees accelerate the staining process, making detection of target nucleic acid faster and more sensitive as compared to individual nanoparticles. Where conductance is increased by gold-promoted reduction of silver or nanoparticle trees, one or just a few individual target analytes present in a sample can be detected.

The chip could be readily incorporated into other environments including a microfluidic cartridge platform (plastic or otherwise), heating elements, or circuit boards.

### EXAMPLES

The following are examples of electrical detection of specific binding events using known oligonucleotides.

### Example 1

(Gold Probe Concentration Study):

1. Gold nanoparticle probes were prepared as described in U.S. Pat. No. 6,506,564, which is hereby fully incorporated by reference. The oligonucleotide sequence used was a repeating sequence of 20 A's.

2. Prepare aliquots of the following gold probe concentrations: 10 fM, 100 fM, 1 pM, 10 pM, 100 pM, 1 nM.

3. Clean the chip with 0.2% SDS solution for 5 minutes and flush with Nanopure water. Spin Dry. Dip in absolute ethanol for 1 minute and spin dry.

4. Approximately 1 mL of Poly-L-lysine (0.01% “Stock” Solution (Sigma 25988-63-0) was applied directly onto 4 of 9 chips with a pipetter and rotated at low speed for 30 minutes.

5. Attach a Dow Corning Sylgard 184 gasket that includes “wells” that hold 4 μL over each of 9 electrode pairs on a chip. The gasket allows a uniform spot shape and prevents cross-contamination.

6. Spot 4 μL of each concentration on each electrode (5 total electrodes). Spare up to three electrodes for a “Negative Control” (NC).

7. Allow the chips to incubate in a plastic pipetter tray containing moist Kim-wipes for 1 hour.

8. Using a silver developer solution, such as Sigma (St. Louis, Mo.) Silver Enhancement Solution A (Part #S-5020) and Enhancement Solution B (Part #S-5145) mixed in a 1:1 ratio, apply silver developer to the entire chip and develop for 2 min on a shaker plate at low speed or by manually shaking the Petri dish.

9. Gently quench the developer and chip in a water bath, spin dry, and record the resistance for each electrode.

10. Repeat step 7 until a signal has developed for each electrode.

In this study, resistance changes between electrodes after binding of gold nanoparticle probes resulted in a resistance change from about 5×10⁸Ω to as low as 1KΩ, depending on the concentration of gold probes used. Optimal increase in conductivity vs. silver development time varied from about 12 minutes to about 16 minutes, again depending on the concentration of gold probes.

### Example 2

(Surface Evaluation/Two-Point Mutation Sequences):

1. Silylated Chips (referred to as “Untreated”) were prepared as follows:

Chips were cleaned with 0.2% SDS solution, water and ethanol, and dried.

Silylated Oligonucleotide capture strands (20 μM concentration) were manually spotted in 2 μLiter droplets using a manual pipetter. The capture strands had the following sequences:

5′ TGA AAT TGT TAT C PegPegPeg 3′ (Positive Control Capture Strand)

5′ TGA AAG GGT TAT C PegPegPeg 3′ (Mutant Capture Strand) The Probe had a complementary sequence to the Positive Control Capture:

3′ Epi-A20-GAT AAC AAT TTC A

2. Silane-modified chips (referred to as “Treated”) were prepared as follows:

Chips were soaked in 5% Isocyanate in absolute EtOH for 1 hour and then dried.

Amine-modified oligonucleotide capture strands (20 μM concentration) were manually spotted in 2 μLiter droplets using a manual pipetter. The capture strands had the following sequence:

5′TGA AAT TGT TAT C PegPegPeg 3′ (Positive Control Capture Strand)

5′TGA AAG GGT TAT C PegPegPeg 3′ (Mutant Capture Strand) The Probe had a complementary sequence to the Positive Control Capture:

3′ Epi-A20-GAT AAC AAT TTC A

In each case, three electrodes were spotted with “Positive Control” capture strands which correspond with the matching Probe sequence, and three electrodes were spotted with a “Mutant” Capture strand which differed in two base pairs from the same matching Probe sequence.

The remaining electrodes were not spotted, and were thus “Negative” Controls.

Chips were hybridized with 10 nM positive control probe at 40 degrees C. for 2 hours.

Total Silver development time was 9 minutes in three-minute increments.

In this study, resistance changes between electrodes after binding of gold nanoparticle probes resulted in a resistance change from about 5×10⁸Ω to as low as about 100Ω after about 40 minutes of silver development. The mutant captures did not show a measurable change in resistance, and two of three negative controls also did not show a measurable change. A third electrode for the negative control was defective, and showed a constant resistance of about 100kΩ.

### Example 3

(Factor V Study):

1. Pre-treatment and chip preparation is same as Two-Point Mutation/Surface Evaluation study.

2. Glass (Pyrex) substrate chips (both “Treated” Isocyanate, and “Untreated” Silylated) were spotted with Factor V Wild Type, Prothrombin, negative Control, and positive Control sequences. The concentration of oligonucleotides spotted was 20μM, and the sequences were as follows:

Capture strand: Wild Type Factor V

Label: Factor V 43H

Sequence: GGC GAG GAA TA-(peg)3-NH2

Capture Strand: Positive Control

Label: PHA2H

Sequence: TGA AAT TGT TAT C-(peg)3-NH2

Capture Strand: Negative Control

Sequence: ACT TTA ACA ATA G-(peg)3-NH2

Length: 13

Capture strand: Wild Type Prothrombin

Label: PRO 19H

Sequence: CTC GCT GAG AG-(peg)3-NH2

1. PCR quantities of Factor V Wild Type target are used with 10 nM concentration of gold probes during hybridization. The gold probes were prepared as described in example 1 above.

2. Hybridization time was 30 minutes at 38 degrees C.

3. Total silver development time was 9 minutes in units of three minutes.

In this study, resistance changes indicating the presence of Factor V Wild Type occurred in 9 minutes, with at least a 100 fold difference in signal intensity between the negative control and Wild Type signal between electrodes.

It should be understood that the illustrated embodiments are exemplary only and should not be taken as limiting the scope of the present invention. The claims should not be read as limited to the described order or elements unless stated to that effect. Therefore, all embodiments that come within the scope of the following claims and equivalents thereto are claimed as the invention.

We claim:

1. An apparatus for electrically detecting at least one first target analyte in a sample, the first target analyte having at least a first binding site and a second binding site, the apparatus comprising:
a substrate having at least a first and a second patterned conductor on its surface, the first patterned conductor being separated from the second patterned conductor, wherein the patterns of the first patterned conductor and the second patterned conductor form at least two substantially non-conducting gaps between the first patterned conductor and the second patterned conductor; and
at least one capture probe that specifically binds to the first binding site of the first target analyte, the at least one capture probe being immobilized on the surface of the substrate within at least one of the substantially non-conducting gaps;
wherein the presence of at least one detection conjugate bound to the first target analyte which is in turn bound to the at least one capture probe, is electrically detectable.

2. The apparatus of claim 1, wherein the patterned conductors are interdigitated.

3. The apparatus of claim 2, wherein the interdigitated conductors and the at least two substantially non-conducting gaps create a pattern covering between about 0.5 square millimeters and about 2 square millimeters of the substrate.

4. The apparatus of claim 1, wherein each of the at least two substantially non-conducting gaps are between about 10 microns and about 100 microns wide and are substantially linear.

5. The apparatus of claim 1, wherein the substrate further comprises a patterned insulator between the first patterned conductor and the second patterned conductor.

6. The apparatus of claim 1, wherein each detection conjugate comprises:
a gold nanoparticle; and
a probe attached to the gold nanoparticle, the probe specifically bindable to the second binding site of the first target analyte.

7. The apparatus of claim 1, wherein each detection conjugate comprises:
a label; and
a probe attached to the label, the probe specifically bindable to the second binding site of the first target analyte.

8. The apparatus of claim 6, wherein the electrical detection further comprises detecting silver aggregated on the gold nanoparticle.

9. The apparatus of claim 1, wherein the electrical detection comprises measuring the conductivity between the first patterned conductor and the second patterned conductor.

10. The apparatus of claim 8, wherein the electrical detection comprises measuring the conductivity between the first patterned conductor and the second patterned conductor.

11. The apparatus of claim 1, wherein the apparatus is further capable of detecting at least one second target analyte in the sample, the second target analyte having at least a first binding site and a second binding site, the apparatus further comprising:
at least a third and a fourth patterned conductor on the surface of the substrate, the third patterned conductor being separated from the fourth patterned conductor, wherein the patterns of the third patterned conductor and the fourth patterned conductor form at least two substantially non-conducting gaps between the third patterned conductor and the fourth patterned conductor; and
at least one second capture probe that specifically binds to the first binding site of the second target analyte, the at least one second capture probe being immobilized on the surface of the substrate within the at least two substantially non-conducting gaps between the third patterned conductor and the fourth patterned conductor;
wherein the presence of at least one detection conjugate bound to the second target analyte which is in turn bound to the second capture probe is electrically detectable.

12. The apparatus of claim 1, wherein the detection conjugate comprises:
a particle; and
a probe attached to the particle, the probe specifically bindable to the second binding site of the first target analyte;
wherein the detection conjugate, the first target analyte, and the at least one capture probe form a complex that creates an electrically detectable change between the first patterned conductor and the second patterned conductor when the first target analyte and the detection conjugate are contacted with the at least one capture probe under conditions effective to allow for specific binding interactions between the at least one capture probe and the first target analyte and between the first target analyte and the detection conjugate.

13. An apparatus for electrically detecting at least one first nucleic acid in a sample, the first nucleic acid having at least a first binding site and a second binding site, the apparatus comprising:
a substrate having at least a first and a second patterned conductor on its surface, the first patterned conductor being separated from the second patterned conductor, wherein the patterns of the first patterned conductor and the second patterned conductor form at least two substantially non-conducting gaps between the first patterned conductor and the second patterned conductor; and
at least one capture probe that specifically binds to the first binding site of the first nucleic acid, the at least one capture probe being bound to the surface of the substrate within the at least one of the two substantially non-conducting gaps;
wherein the presence of at least one detection conjugate bound to the first nucleic acid which is in turn bound to the at least one capture probe, is electrically detectable.

14. The apparatus of claim 13, wherein the patterned conductors are interdigitated.

15. The apparatus of claim 14, wherein the interdigitated conductors and the at least two substantially non-conducting gaps create a pattern covering between about 0.5 square millimeters and about 2 square millimeters of the substrate.

16. The apparatus of claim 13, wherein each of the at least two substantially non-conducting gaps are between about 10 microns and about 100 microns wide and are substantially linear.

17. The apparatus of claim 13, wherein the substrate further comprises a patterned insulator between the first patterned conductor and the second patterned conductor.

18. The apparatus of claim 13, wherein the electrical detection further comprises detecting silver aggregated on the detection conjugate.

19. The apparatus of claim 18, wherein the electrical detection comprises measuring the conductivity between the first patterned conductor and the second patterned conductor.

20. The apparatus of claim 13, wherein the apparatus is further capable of detecting at least one second nucleic acid in the sample, the second nucleic acid having at least a first binding site and a second binding site, the apparatus further comprising:
at least a third and a fourth patterned conductor on the surface of the substrate, the third patterned conductor being separated from the fourth patterned conductor, wherein the patterns of the third patterned conductor and the fourth patterned conductor form at least two substantially non-conducting gaps between the third patterned conductor and the fourth patterned conductor; and
at least one second capture probe that specifically binds to the first binding site of the second nucleic acid, the at least one second capture probe being bound to the surface of the substrate within the at least two substantially non-conducting gaps between the third patterned conductor and the fourth patterned conductor;
wherein the presence of at least one detection conjugate bound to the second nucleic acid which is in turn bound to the at least one second capture probe, is electrically detectable.

21. An apparatus for detecting at least a first target analyte and a second target analyte in a sample, the first target analyte having at least a first binding site and a second binding site and the second target analyte having at least a first binding site and a second binding site, the apparatus comprising:
a substrate that includes:
a first and a second patterned conductor on its surface, the first patterned conductor being separated from the second patterned conductor, wherein the patterns of the first patterned conductor and the second patterned conductor form at least two substantially non-conducting gaps between the first patterned conductor and the second patterned conductor; and
a third and a fourth patterned conductor on its surface, the third patterned conductor being separated from the fourth patterned conductor, wherein the patterns of the third patterned conductor and the fourth patterned conductor form at least two substantially non-conducting gaps between the third patterned conductor and the fourth patterned conductor;
wherein the first patterned conductor and the second patterned conductor together comprise a first detection region and the third patterned conductor and the fourth patterned conductor together comprise a second detection region; and
wherein the presence of the first target analyte and the second target analyte in the first and the second detection region, respectively, is electrically detectable when the first binding sites of the target analytes are bound to detection conjugates comprising electrically conductive particles and the second binding sites of the target analytes are bound to first capture probes attachable to the first detection region and to second capture probes attachable to the second detection region.

22. The apparatus of claim 21, wherein the patterned conductors are interdigitated.

23. The apparatus of claim 22, wherein the first detection region and the second detection region each create a pattern covering between about 0.5 square millimeters and about 2 square millimeters of the substrate.

24. The apparatus of claim 21, wherein each of the substantially non-conducting gaps are between about 10 microns and about 100 microns wide and are substantially linear.

25. The apparatus of claim 21, wherein the substrate further comprises a patterned insulator between the first patterned conductor and the second patterned conductor.

26. The apparatus of claim 21, wherein the electrical detection further comprises detecting silver aggregated on the electrically conductive particles.

27. The apparatus of claim 26, wherein the electrical detection comprises measuring the conductivity between the first patterned conductor and the second patterned conductor.

28. A method for detecting a target analyte having a first binding site and a second binding site, the method comprising:
(a) providing a substrate having at least a first and a second patterned conductor, the first patterned conductor being separated from the second patterned conductor, wherein the patterns of the first patterned conductor and the second patterned conductor form at least two substantially non-conducting gaps;
(b) contacting, to the substrate, at least one capture probe that specifically binds to the first binding site of the target analyte, the at least one capture probe being immobilized on the surface of the substrate within at least one of the two substantially non-conducting gaps oligonucleotides complementary to the first binding site of the target analyte;
(c) providing at least one detection conjugate comprising:
an electrically conductive particle; and
a probe attached to the electrically conductive particle, the probe specifically bindable to the second binding site of the target analyte; and
(d) contacting the substrate and the at least one detection conjugate provided in (a) and (c), respectively, with the target analyte under hybridizing conditions to bind the target analyte to the at least one capture probe and to bind the at least one detection conjugate to the target analyte; and
(e) electrically detecting the at least one detection conjugate bound to the target analyte which is in turn bound to the at least one capture probe.

29. The method of claim 28, wherein the electrically conductive particles are gold nanoparticles.

30. The method of claim 28, wherein the electrically conductive particles are selected from the group consisting of: silver nanoparticles and silver aggregated with gold nanoparticles.

31. The method of claim 28, wherein the patterned conductors are interdigitated.

32. The method of claim 28, wherein the capture probes are applied to the substrate by a robotic arrayer.

33. The method of claim 31, wherein the interdigitated conductors and the at least two substantially non-conducting gaps create a pattern covering between about 0.5 and about 2 square millimeters of the substrate.

34. The method of claim 28, wherein each of the at least two substantially non-conducting gaps are between about 10 microns and about 100 microns wide and are substantially linear.

35. The method of claim 28, wherein the substrate further comprises a patterned insulator between the first patterned conductor and the second patterned conductor.

36. The method of claim 28, wherein the target analyte is RNA or DNA.

37. The method of claim 28, wherein the target analyte is of human, bacterial, viral, or fungal origin.

38. The method of claim 28, wherein the target analyte is a gene associated with a disease.

39. The method of claim 28, wherein the target analyte is a synthetic DNA, a synthetic RNA, a structurally modified natural or synthetic RNA, or a structurally modified natural or synthetic DNA.

40. The method of claim 28, wherein the electrical detection is enhanced by silver deposition of the electrically conductive nanoparticles.

41. The method of claim 28, wherein the electrical detection comprises measuring the conductivity between the first patterned conductor and the second patterned conductor.

42. The method of claim 28, wherein the at least one detection conjugate further comprises a probe that specifically binds to a binding site of a second type of detection conjugate that further comprises an electrically conductive nanoparticle, the method further comprising:
contacting the at least one detection conjugate with at least one detection conjugate of the second type; and
electrically detecting the second type of detection conjugate bound to the target analyte bound to the substrate.

43. The method of claim 42, wherein the electrical detection is enhanced by silver deposition of the electrically conductive nanoparticles.

44. A method for detecting a nucleic acid having a first binding site and a second binding site, the method comprising:
providing a substrate having a plurality of patterned conductors arranged in complementary pairs, the first patterned conductor of any complementary pair of conductors being separated from the second patterned conductor of that pair, wherein the patterns of the first patterned conductor and the second patterned conductor of each pair forms at least two substantially linear, substantially non-conducting gaps, and wherein each complementary pair of conductors covers at least one square millimeter of the substrate;
robotically contacting to the substrate oligonucleotides complementary to the first binding site of the nucleic acid;
providing gold nanoparticles having bound thereto oligonucleotides complementary to the second binding site of the nucleic acid;
contacting the substrate and the gold nanoparticles provided in (a) and (c), respectively, with the nucleic acid under hybridizing conditions to bind the nucleic acid to the substrate and to the gold nanoparticles;
silver staining the gold nanoparticles; and
electrically detecting the silver-stained gold nanoparticles bound to the nucleic acid which is in turn bound to the substrate by measuring a change in conductance between pairs of patterned conductors.

45. The method of claim 44, wherein the patterned conductors are interdigitated.

46. The method of claim 45, wherein the interdigitated conductors and the at least two substantially non-conducting gaps create a pattern covering between about 0.5 square millimeters and about 2 square millimeters of the substrate.

47. The method of claim 44, wherein each of the at least two substantially non-conducting gaps are between about 10 microns and about 100 microns wide and are substantially linear.

48. The method of claim 44, wherein the substrate further comprises a patterned insulator between the first patterned conductor and the second patterned conductor.

49. The method of claim 44, wherein the electrical detection further comprises detecting silver aggregated on the gold nanoparticles.

50. The method of claim 49, wherein the electrical detection comprises measuring the conductivity between the first patterned conductor and the second patterned conductor.

51. A method of detecting a nucleic acid having at least two binding sites, the method comprising:
(a) contacting a nucleic acid with a substrate having oligonucleotides attached thereto, the oligonucleotides being located between a first and a second patterned electrode;
wherein the patterns of the first patterned electrode and the second patterned electrode form at least two substantially non-conducting gaps between the first patterned electrode and the second patterned electrode;
the oligonucleotides having a sequence complementary to a first binding site of the sequence of said nucleic acid, the contacting taking place under conditions effective to allow hybridization of the oligonucleotides on the substrate with said nucleic acid;
(b) contacting said nucleic acid bound to the substrate with a first type of labels, the labels being made of a material which can conduct electricity, the labels having one or more types of oligonucleotides attached thereto, at least one of the types of oligonucleotides having a sequence complementary to a second binding site of the sequence of said nucleic acid, the contacting taking place under conditions effective to allow hybridization of the oligonucleotides on the labels with said nucleic acid so as to form a test substrate having labels complexed thereto;
(c) contacting the test substrate with an aqueous salt solution having a salt concentration effective to sufficiently remove non-specifically bound labels; and
(d) detecting an observable change.

52. The method of claim 51, wherein detecting an observable change includes detecting a change in an electrical property between the first patterned electrode and the second patterned electrode and the change in the electrical property between the first patterned electrode and the second patterned electrode includes a change in conductivity, resistivity, capacitance, or impedance.

53. The method of claim 51, wherein the substrate has a plurality of pairs of electrodes located on it in an array to allow for the detection of multiple portions of a single nucleic acid, the detection of multiple different nucleic acids, or both, each of the pairs of electrodes having a type of oligonucleotides attached to the substrate between them.

54. The method of claim 51, wherein the labels are made of metal.

55. The method of claim 51, wherein the labels comprise nanoparticles.

56. The method of claim 51, wherein the labels comprise metallic or semiconductor nanoparticles.

57. The method of claim 51, wherein the labels comprise gold nanoparticles.

58. The method of claim 51, wherein the substrate is contacted with silver stain to produce the change in conductivity.

59. The method of claim 51, further comprising:
(d) contacting the first type of labels bound to the substrate with a second type of labels, the labels being made of a material which can conduct electricity, the labels having oligonucleotides attached thereto, at least one of the types of oligonucleotides on the second type of labels comprising a sequence complementary to the sequence of one of the types of oligonucleotides on the first type of labels, the contacting taking place under conditions effective to allow hybridization of the oligonucleotides on the first and second types of labels; and
(e) detecting the change in an electrical property between the first patterned electrode and the second patterned electrode.

60. The method of claim 59, wherein the change in an electrical property between the first patterned electrode and the second patterned electrode includes a change in conductivity, resistivity, capacitance, or impedance.

61. The method of claim 59, wherein at least one of the types of oligonucleotides on the first type of labels has a sequence complementary to the sequence of at least one of the types of oligonucleotides on the second type of labels and the method further comprises:
(f) contacting the second type of labels bound to the substrate with the first type of labels, the contacting taking place under conditions effective to allow hybridization of the oligonucleotides on the first and second types of labels; and
(g) detecting the change in an electrical property between the first patterned electrode and the second patterned electrode.

62. The method of claim 61, wherein the change in the electrical property between the first patterned electrode and the second patterned electrode includes a change in conductivity, resistivity, capacitance, or impedance.

63. The method of claim 61, wherein step (d) or steps (d) and (f) are repeated one or more times and the change in conductivity is detected.

64. The method of claim 51, further comprising:
(d) contacting the first type of labels bound to the substrate with an aggregate probe having oligonucleotides attached thereto, the labels of the aggregate probe being made of a material which can conduct electricity, at least one of the types of oligonucleotides on the aggregate probe comprising a sequence complementary to the sequence of one of the types of oligonucleotides on the first type of labels, the contacting taking place under conditions effective to allow hybridization of the oligonucleotides on the aggregate probe with the oligonucleotides on the first type of labels; and
(e) detecting the change in an electrical property between the first patterned electrode and the second patterned electrode.

65. The method of claim 64, wherein the change in the electrical property between the first patterned electrode and the second patterned electrode includes a change in conductivity, resistivity, capacitance, or impedance.

66. The method of claim 51, wherein the aqueous salt solution comprises a salt selected from the group consisting of sodium chloride, magnesium chloride, potassium chloride, ammonium chloride, sodium acetate, ammonium acetate, a combination of two or more of these salts, one of these salts in a phosphate buffer, and a combination of two or more of these salts in a phosphate buffer.

67. The method of claim 66, wherein the salt solution is sodium chloride in a phosphate buffer.

68. The method of claim 67, wherein the aqueous salt solution comprises between about 0 M to 0.5 M sodium chloride and between about 0.01 mM to 15 mM phosphate buffer at pH 7.

69. The method of claim 67, wherein the aqueous salt solution comprises between about 0.005 to 0.1 M sodium chloride and about 10 mM phosphate buffer at pH 7.

70. The method of claim 51, wherein the observing a detectable change comprises determining whether hybridization has occurred.

71. In a method for increasing stringency of hybridization that employs a substrate having at least a first and a second patterned conductor on its surface, the first patterned conductor being separated from the second patterned conductor, wherein the patterns of the first and second patterned conductors for at least two substantially non-conducting gaps between the first patterned conductor and the second patterned conductor, and the substrate having bound capture oligonucleotide probes within at least one of the substantially non-conducting gaps and labeled oligonucleotide detection probes for capturing and detecting one or more target nucleic acids in a sample by hybridization interactions, the sample including nucleic acids having a mismatched base, the improvement comprising including a step of washing the substrate having a hybridized complex of capture probes, target nucleic acid, and detection probes with an aqueous salt solution.

72. The method of claim 71, wherein the aqueous salt solution comprises a salt selected from the group consisting of sodium chloride, magnesium chloride, potassium chloride, ammonium chloride, sodium acetate, ammonium acetate, a combination of two or more of these salts, one of these salts in a phosphate buffer, and a combination of two or more of these salts in a phosphate buffer.

73. The method of claim 72, wherein the salt solution is sodium chloride in a phosphate buffer.

74. The method of claim 73, wherein the aqueous salt solution comprises between about 0 M to 0.5 M sodium chloride and between about 0.01 mM to 15 mM phosphate buffer at pH 7.

75. The method of claim 72, wherein the aqueous salt solution comprises between about 0.005 to 0.1 M sodium chloride and about 10 mM phosphate buffer at pH 7.

76. The method of claim 71, wherein the detection probes comprise nanoparticle-oligonucleotide conjugates.

77. The method of claim 71, wherein the conjugates are gold nanoparticle-oligonucleotide conjugates.
